(19) United States
(12) Patent Application Publication
(10) Pub. No.: US 2020/0046339 A1
(43) Pub. Date: Feb. 13, 2020

(54) THREE-IN-ONE SUTURING DEVICE FOR CLOSING CANNULA HOLE

(71) Applicant: Shenzhen People's Hospital (Shenzhen Institute of Respiratory Disease), Shenzhen (CN)

(72) Inventors: Kai Pan, Shenzhen (CN); Xuefei Yang, Shenzhen (CN); Chang Zhu, Shenzhen (CN)

(21) Appl. No.: 16/357,429

(22) Filed: Mar. 19, 2019

(30) Foreign Application Priority Data
Aug. 13, 2018 (CN) 201821294726.0

(51) Int. Cl. A61B 17/04; A61B 17/06

(52) U.S. Cl. CPC A61B 17/0469; A61B 17/0482; A61B 17/0625; A61B 17/0485; A61B 17/06066

(57) ABSTRACT

Disclosed is a three-in-one suturing device for closing a cannula hole, belonging to the field of medical instruments and solving the troublesome suturing problem in laparoscopic surgery. The cannula hole is closed in a quick, effective and safe manner by means of the cooperation of a pull hook, a suture needle and a fishbone thread. In the entire threading process, the needle tip only needs to penetrate the human body without going out of the human body; therefore, the operation difficulty and time of the cannula hole suturing surgery are reduced; and the fishbone thread is used to suture the cannula hole from the bottom of the suture portion.

### CROSS REFERENCE TO RELATED APPLICATIONS

The present application claims the benefit of Chinese Patent Application No. 201821294726.0 filed on Aug. 13, 2018, the contents of which are incorporated herein by reference in its entirety.

### BACKGROUND OF THE INVENTION

### 1. Technical Field

The invention belongs to the field of medical instruments and is used for suturing a cannula hole in laparoscopic surgery. The cannula hole is closed in a quick, effective and safe manner by means of the cooperation of a pull hook, a suture needle and a fishbone thread.

### 2. Description of Related Art

Laparoscopic surgery is a newly developed minimally invasive method and a trend in the development of modern surgical methods. With the rapid advancement of industrial manufacturing technology, the integration of related disciplines has laid a firm foundation for the development of new technologies and methods. Moreover, due to the increasingly skilled operation of doctors, many traditional open surgical procedures have now been replaced by endoscopic procedures, greatly increasing the chances of surgery. Laparoscopic and minimally invasive surgery is more popular with patients than the traditional surgery; especially for young patients, the laparoscopic and minimally invasive surgery is more likely to be accepted because postoperative scars are small and the aesthetic requirements are met. The minimally invasive surgery is the general trend and goal of surgical development. At present, most operations can be performed by laparoscopy, such as appendectomy, gastric and duodenal ulcer perforation repair, hernia repair, colectomy, splenectomy, adrenalectomy, ovarian cyst removal, ectopic pregnancy, hysterectomy, etc.; with the improvement of laparoscopic techniques and the improvement of the operation level of laparoscopic doctors, laparoscopy can be used in almost all surgical procedures.

In a laparoscopic surgery, 3-5 0.5-1 cm small incisions need to be made in the abdomen of the patient to place cannulas, and the operations in the surgery are all performed through the cannula holes; one of the cannula holes is used to place a laparoscope and the cannula hole holding the laparoscope is generally located in the umbilicus; after the surgery is completed, the cannula hole in the umbilicus cannot automatically heal like other cannula holes and needs to be sutured; usually, a suture needle is directly used to pull a suture thread to penetrate back and forth through the portion to be sutured, thus realizing suturing of the cannula hole. Since the cannula hole has a small diameter and the suture position is deep, the operation is very inconvenient, especially in patients who are obese, it is difficult to suture the bottom layer of the cannula hole, and the suture effect is poor. It is prone to cannula hole hernia.

### BRIEF SUMMARY OF THE INVENTION

Directed to the above clinical problems, the invention provides a three-in-one suturing device to close the cannula hole in a quick, effective and safe manner by means of the cooperation of a pull hook, a suture needle and a fishbone thread.

The three-in-one suturing device for closing a cannula hole comprises: a pull hook, which is composed of a grip portion and an attaching portion having a through hole in the middle, wherein the upper surface of the attaching portion is provided with a raised serration, the lower surface of the attaching portion is fixedly connected with a protective shell, and the middle through hole is covered with a layer of elastic film; a suture needle having a groove at the front end; and a fishbone thread, comprising a thread body that can be caught in the groove and barbs that can be attached to the film, wherein after being caught in the groove, the fishbone thread pierces the film along with the suture needle, and when the suture needle leaves the film in the original direction, the barbs are attached to the film, and then the fishbone thread is detached from the groove and kept in the protective shell, and moves along with the pull hook.

The grip portion forms a right angle with the attaching portion; the end of the attaching portion, away from the grip portion, is bent upward to form a hooking portion; when the grip portion is lifted up, the hooking portion fixes a hooked object; when the grip portion is lowered, the hooked object is separated from the hooking portion.

Wherein, the raised serration forms an acute angle with the attaching portion; when the grip portion is lifted up, the raised serration fixes the hooked object; when the grip portion is lowered, the hooked object is separated from the raised serration.

The suture needle comprises a needle tip and a needle body; the suture needle is gradually sharpened in a direction from the needle body to the needle tip; the groove is formed in the needle tip segment and the opening of the groove faces the needle tip.

The barbs fixedly connected to the thread body form acute angle grooves and obtuse angle grooves with the thread body; when the fishbone thread enters the film, the openings of the obtuse angle grooves face the film; when the fishbone thread leaves the film, the openings of the acute angle grooves face the film; therefore, the fishbone thread can enter the film easily but can hardly go out of the film.

The outer surface of the protective shell is a smooth curved surface, so that the pull hook can come into and go out of the cannula hole easily and the suture needle can be isolated from the tissues in the abdominal cavity.

The invention has the advantages that: compared with the prior art, the upper surface of the attaching portion of the pull hook is provided with a raised serration; during an operation, the pull hook is lifted up, the attaching portion and the portion to be sutured abut against each other, the raised serration tightly catches the portion to be sutured, and thus the portion to be sutured does not slide out from the attaching portion. The front end of the suture needle is provided with a groove, and the opening of the groove faces the sharp end of the needle; the fishbone thread is pushed by the side wall of the groove and is kept in the groove when pierced into the protective shell along with the suture needle; when the suture needle leaves along the original path, the barbs of the fishbone thread are attached to the elastic film, so that the fishbone thread remains on the pull hook and moves away from the body along with the pull hook. The lower surface of the attaching portion is fixedly connected to the protective shell, and after penetrating through the through hole, the suture needle is blocked by the protective shell and will not accidentally injure organs inside the human body; the outer surface of the protective shell is smooth, so it is easier to enter and exit the human body. During the suturing process, the suture needle only needs to be pierced into the human body without going out from the human body, the fishbone thread can leave the human body along with the pull hook. The problem that the suture needle in the traditional surgery is difficult to go out from the human body can be overcome; the difficulty of the surgery is lowered, and the fishbone thread is used to suture the cannula hole from the bottom of the portion to be sutured.

### BRIEF DESCRIPTION OF THE SEVERAL VIEWS OF THE DRAWINGS

FIG. 1 is a structural view of a pull hook of the invention;

FIG. 2 is a structural view of a suture needle of the invention;

FIG. 3 is a structural view of a fishbone thread of the invention;

FIG. 4 is a schematic view of the suturing process of the invention; and

FIG. 5 is a schematic view of the invention after suturing.

Reference numerals for main components are described as follows:

1, pull hook; 2, fishbone thread; 3, suture needle; 4, first portion to be sutured; 5, second portion to be sutured; 11, grip portion; 12, attaching portion; 13, through hole; 14, raised serration; 15, hooking portion; 16, anti-slip portion; 17, protective shell; 21, barb; 22, thread body; 31, needle tip; 32, needle body; 33, groove.

### DETAILED DESCRIPTION OF THE INVENTION

In order to more clearly illustrate the invention, the invention will be further described below in conjunction with the accompanying drawings.

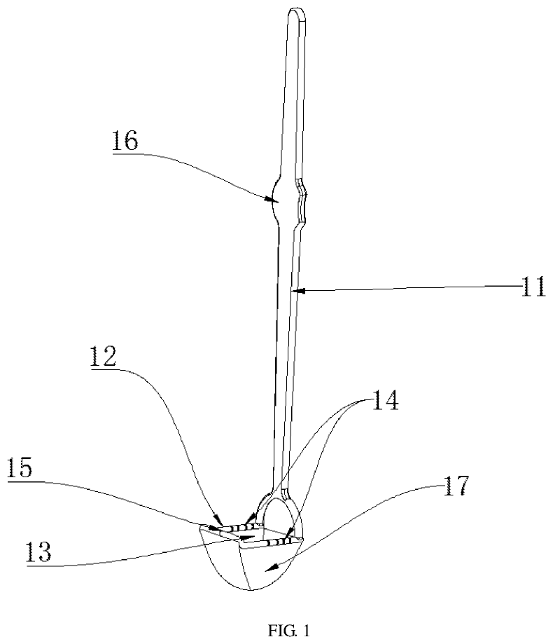

Referring to FIG. 1, shown is a three-in-one suturing device for closing a cannula hole, comprising a pull hook 1, a suture needle 3 and a fishbone thread 2; the pull hook 1 comprises a grip portion 11 and an attaching portion 12 having a through hole 13 in the middle. The upper surface of the attaching portion 12 is provided with a raised serration 14, the lower surface of the attaching portion 12 is fixedly connected to a protective shell 17, and the middle through hole 13 is covered with a layer of elastic film; the suture needle 3 is configured to pierce the film and has a groove 33; the fishbone thread 2 comprises a thread body 22 that can be caught in the groove 33 and barbs 21 that can be attached to the film; after being caught in the groove 33, the fishbone thread 2 pierces the film along with the suture needle 3, and when the suture needle 3 leaves the film in the original direction, the barbs 21 are attached to the film, and then the fishbone thread 2 is detached from the groove 33 and kept in the protective shell 17, and moves along with the pull hook 1.

In this embodiment, the grip portion 11 is provided with protruding anti-slip portions 16 on both sides thereof, and the anti-slip portions 16 are integrally formed in an “M” shape in which the two ends are raised and the middle is recessed, and fingers hold the middle recess to prevent the pull hook 1 from being slip down in the hand; the grip portion 11 forms a right angle with the attaching portion 12; the end of the attaching portion 12, away from the grip portion 11, is bent upward to form a hooking portion 15; when the grip portion 11 is lifted up, the hooking portion 15 fixes a hooked object; when the grip portion 11 is lowered, the hooked object is separated from the hooking portion 15; the raised serration 14 forms an acute angle with the attaching portion 12; when the grip portion 11 is lifted up, the raised serration 14 fixes the hooked object; when the grip portion 11 is lowered, the hooked object is separated from the raised serration 14. During an operation, the pull hook 1 is lifted up, the hooking portion 15 and the raised serration 14 work together to hook the portion to be sutured to fix the position of the portion to be sutured; when the suture needle 3 penetrates through the portion to be sutured and then is inserted in the through hole 13 of the pull hook 1, the protective shell 17 blocks the suture needle 3 to prevent the suture needle 3 from accidentally injuring the organs in the human body during the operation; the outer surface of the protective shell 17 is a smooth curved surface and does not injure the organs in the human body; after the suture needle 3 leaves the sutured portion in the original direction, the fishbone thread 2 remains in the accommodating space; after the pull hook 1 leaves the body from the cannula hole, the fishbone thread 2 follows the pull hook 1 to leave the human body.

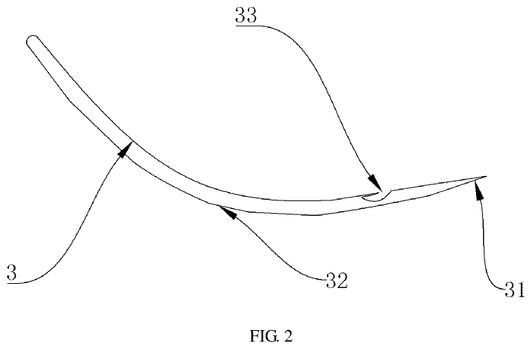

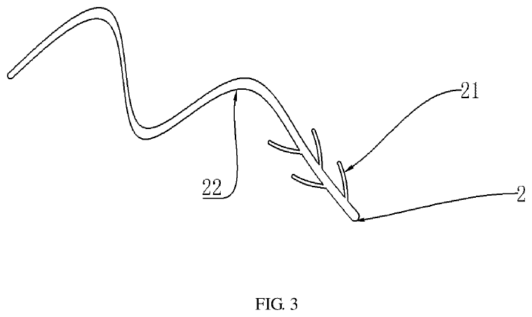

Referring to FIGS. 2 and 3, the suture needle 3 comprises a needle tip 31 and a needle body 32; the suture needle 3 is gradually sharpened in a direction from the needle body 32 to the needle tip 31; the groove 33 is formed in the needle tip 31 and the opening of the groove 33 faces the needle tip 31 end; the fishbone thread 2 is caught in the groove 33, and when the needle tip 31 enters the human body from the portion to be sutured, the bottom of the groove 33 abuts the fishbone thread 2 to drive the fishbone thread 2 to enter the human body together and the suture needle is inserted into the accommodating space; when the needle tip 31 leaves the human body in the original direction, the fishbone thread 2 is separated from the suture needle 3 through the opening of the groove 33 under the action of the resistance of the film; the shape of the suture needle 3 is a smooth curve having only one pole, preferably an arc; the suture needle 3 receives the least resistance when piercing the portion to be sutured; the barbs 21 of the fishbone thread 2 are fixedly connected to the thread body 22, forming acute angle grooves and obtuse angle grooves with the thread body 22; when the fishbone thread enters the film, the openings of the obtuse angle grooves face the film; when the fishbone thread leaves the film, the openings of the acute angle grooves face the film; the barbs 21 are arranged on one side of the thread body 22 or symmetrically arranged on both sides of the main thread body or staggered on both sides of the main thread body, and the openings of the acute angle grooves are all the same, and it is preferred that the barbs 21 are staggered on both sides of the main thread body 22; in this way, the fishbone thread 2 is less resisted by the film when it enters the accommodating space, and is caught when it leaves; the effect that the fishbone thread enters the film easily but hardly goes out of the film is achieved.

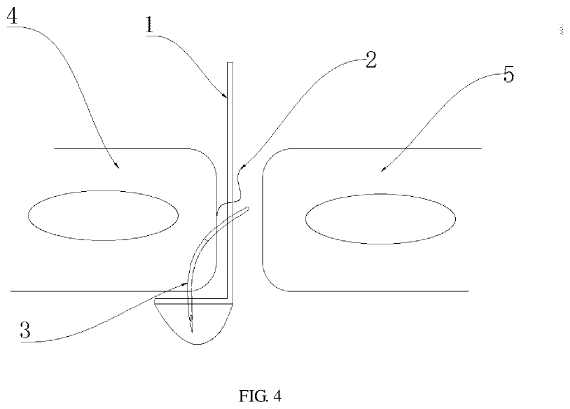

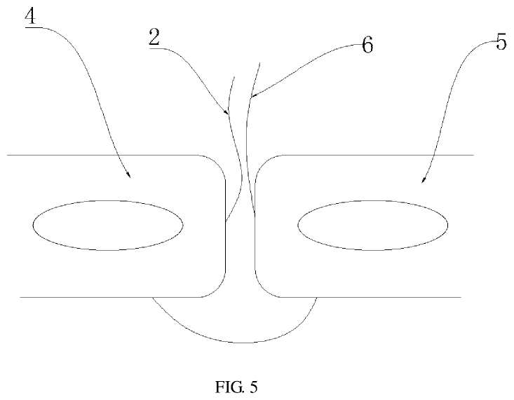

Referring to FIGS. 4 and 5, at the time of surgical suturing, the fishbone thread 2 is firstly caught in the groove 33 of the suture needle 3, so that the openings of the obtuse angle grooves formed by the barbs of the fishbone thread 2 and the thread body 22 face the needle tip 31; after entering a human body, the pull hook 1 sticks to a first portion 4 to be sutured and the grip portion 1 is lifted up so that the hooking portion 15 and the raised serration 14 tightly hook the first portion 4 to be sutured to fix the position of the first portion 4 to be sutured; then, the suture needle 3 is used to drive the fishbone thread 2 to pierce from the upper surface of the first portion 4 to be sutured, go out from the lower surface of the first portion 4 to be sutured, and penetrate through the film; since the openings of the obtuse angle grooves formed by the barbs 21 of the fishbone thread 2 and the thread body 22 face the needle tip 31, at the time of piercing, the barbs 21 are less resisted; and after the piercing, the needle tip 31 is blocked by the protective shell 17 and does not accidentally injure other organs in the human body; and then the suture needle 3 leaves the first portion 4 to be sutured along the original direction, and the barbs 21 of the fishbone thread 2 and the film abut each other, so that the fishbone thread 21 slides out from the opening of the groove 33, remains in the protective shell of the pull hook 1, and goes out of the suture opening along with the pull hook 1, thus leaving the human body. the entire process completes the operation that the fishbone thread 2 enters from the upper surface of the first portion 4 to be sutured, penetrates through the lower surface, and then goes back to the upper surface; then, another fishbone thread 6 is caught into the groove 33, the above operation is repeated, so that the other fishbone thread 6 enters from the upper surface of a second portion 5 to be sutured, penetrates through the lower surface, and then goes back to the upper surface; and finally the two fishbone threads 2 located outside the body and the end of the other fishbone thread 6 on the lower surface are overlapped, and then the fishbone thread 2 on the upper surface and the end of the other fishbone thread 6 are pulled upwards at the same time and then overlapped together to complete the suture. During the entire operation, the suture needle 3 is only required to be pierced from top to bottom, and the process of piercing the suture needle 3 from bottom to top during suturing is avoided, which greatly reduces the difficulty of operation; and after suturing, the suture portion is sutured with the fishbone thread from the lower surface of the cannula hole, achieving a good suturing effect.

The invention has the following advantages:

1. The upper surface of the attaching portion of the pull hook is provided with a raised serration; during an operation, the pull hook is lifted up, the attaching portion and the portion to be sutured abut against each other, the raised serration tightly catches the portion to be sutured, and thus the portion to be sutured does not slide out from the attaching portion.

2. The front end of the suture needle is provided with a groove, and the opening of the groove faces the sharp end of the needle; the fishbone thread is pushed by the side wall of the groove and is kept in the groove when pierced into the protective shell along with the suture needle; when the suture needle leaves, the barbs of the fishbone thread is attached to the elastic film, so that the fishbone thread remains on the pull hook and moves away from the body along with the pull hook.

3. The lower surface of the attaching portion is fixedly connected to the protective shell, and after penetrating through the through hole, the suture needle is blocked by the protective shell and will not accidentally injure organs inside the human body; the outer surface of the protective shell is smooth, so it is easier to enter and exit the human body.

4. During the entire process of the operation, the suture needle only needs to be pierced into the human body without going out from the human body, the fishbone thread can leave the human body along with the pull hook. The problem that the suture needle in the traditional surgery is difficult to go out from the human body can be overcome; the difficulty of the surgery is lowered, and the fishbone thread is used to suture the cannula hole from the bottom of the suture portion.

The above disclosure is only a few specific embodiments of the invention, but the invention is not limited thereto, and any changes that can be conceived of by those skilled in the art should fall within the protection scope of the invention.

What is claimed is:

1. A three-in-one suturing device for closing a cannula hole, comprising:
a pull hook, which is composed of a grip portion and an attaching portion having a through hole in the middle, wherein an upper surface of the attaching portion is provided with a raised serration, a lower surface of the attaching portion is fixedly connected with a protective shell, and the middle through hole is covered with a layer of elastic film;
a suture needle configured to pierce the film and having a groove at a front end; and
a fishbone thread, comprising a thread body that can be caught in the groove and barbs that can be attached to the film,
wherein after being caught in the groove, the fishbone thread pierces the film along with the suture needle, and when the suture needle leaves the film in the original direction, the barbs are attached to the film, and then the fishbone thread is detached from the groove and kept in the protective shell, and moves along with the pull hook.

2. The three-in-one suturing device for closing a cannula hole according to claim 1, wherein the grip portion forms a right angle with the attaching portion; an end of the attaching portion, away from the grip portion, is bent upward to form a hooking portion; when the grip portion is lifted up, the hooking portion fixes a hooked object; when the grip portion is lowered, the hooked object is separated from the hooking portion.

3. The three-in-one suturing device for closing a cannula hole according to claim 1, wherein the raised serration forms an acute angle with the attaching portion; when the grip portion is lifted up, the raised serration fixes the hooked object; when the grip portion is lowered, the hooked object is separated from the raised serration.

4. The three-in-one suturing device for closing a cannula hole according to claim 1, wherein the suture needle comprises a needle tip and a needle body; the suture needle is gradually sharpened in a direction from the needle body to the needle tip; the groove is formed in the needle tip end and openings of the groove faces a sharp end of the needle tip.

5. The three-in-one suturing device for closing a cannula hole according to claim 1, wherein the barbs are fixedly connected to the thread body and form acute angle grooves and obtuse angle grooves with the thread body; when the fishbone thread enters the film, openings of the obtuse angle grooves face the film; when the fishbone thread leaves the film, openings of the acute angle grooves face the film.

6. The three-in-one suturing device for closing a cannula hole according to claim 5, wherein the barbs are arranged on one side of the thread body or symmetrically arranged on both sides of the main thread body or staggered on both sides of the main thread body, and the openings of the acute angle grooves are all the same.

7. The three-in-one suturing device for closing a cannula hole according to claim 4, wherein an outer surface of the protective shell is a smooth curved surface so that the suture needle enters and exits the cannula hole easily, and the protective shell can isolate the abdominal organs when the suture needle penetrates the film of the pull hook, thereby preventing the needle tip accidentally injuring tissues.
